(19) United States
(12) Patent Application Publication
(10) Pub. No.: US 2005/0267092 A1
(43) Pub. Date: Dec. 1, 2005

(54) Novel cephalosporin compounds and process for preparing the same

(76) Inventors: Chang-Seok Lee, Taejeon (KR); Geun-Tae Kim, Taejeon (KR); Yong-Jin Jang, Kyunggi-do (KR); Eun-Jung Ryu, Seoul (KR); Yang-Rae Cho, Taejeon (KR); Hyung-Yeul Joo, Taejeon (KR); Jeong-Eun Shin, Kyunggi-do (KR); Sun-Hwa Lee, Taejeon (KR); Ki-Dong Koo, Seoul (KR)

Correspondence Address: BIRCH STEWART KOLASCH & BIRCH, PO BOX 747, FALLS CHURCH, VA, 22040-0747, US

(21) Appl. No.: 10/505,724

(22) PCT Filed: Feb. 28, 2002
(86) PCT No.: PCT/KR02/00347

§ 371 Date: Oct. 4, 2004

(51) Int. Cl. A61K 31/545; C07D 501/14

(52) U.S. Cl. 514/202; 540/222

(57) ABSTRACT

The present invention relates to a novel cephalosporin compound, and pharmaceutically acceptable non-toxic salt, physiologically hydrolysable ester, hydrate, solvate or isomer thereof, to a pharmaceutical composition comprising the compound, and to a process for preparing the compound.

### TECHNICAL FIELD

The present invention relates to a novel cephalosporin compound useful as an antibiotic agent. More specifically, the present invention relates to a novel cephalosporin compound represented by the following formula (1), which is useful as an antibacterial agent, and particularly, exhibits a potent activity against strains such as methicillin-resistant Staphylococcus aureus (MRSA): 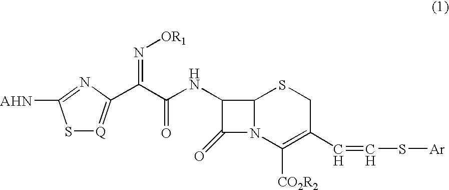
or pharmaceutically acceptable non-toxic salt, physiologically hydrolysable ester, hydrate, solvate or isomer thereof, in which
A represents hydrogen or amino-protecting group,
R₁ represents hydrogen, or represents C₁₋₆ alkyl, C₃₋₄ alkynyl, C₃₋₆ cycloalkyl or C₃₋₆ cycloalkyl-methyl, each of which may comprise one to three atoms selected from the group consisting of oxygen and halogen,
R₂ represents hydrogen or carboxyl-protecting group,
Ar represents 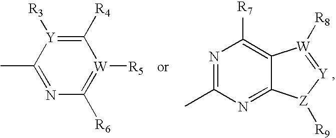
wherein
R₃, R₄, R₅ and R₇ independently of one another represent hydrogen; hydroxyl; C₁₋₆ alkyl; amino which is unsubstituted or substituted by C₁₋₆ alkyl; C₁₋₆ hydroxyalkyl; or C₁₋₆ alkylthio,
R₆ represents hydrogen; hydroxyl; amino which is unsubstituted or substituted by C₁₋₆ alkyl; C₁₋₆ alkyl; or 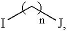
wherein
I represents S, NH, CH₂, or O,
n represents 0, 1, 2, 3, or 4,
J represents amino which is unsubstituted or substituted by C₁₋₆ alkyl; hydroxy; or C₁₋₆ alkoxy,
R₈ and R₉ independently of one another represent hydrogen; C₁₋₆ alkyl; C₁₋₆ alkylamino; hydroxy; or C₁₋₆ alkoxy,
W and Y independently of one another represent N or C, provided that R₃, R₅, R₈ do not exist when W or Y represents N,
Z represents CH or N,
Q represents CH, C—G, or N, wherein G represents halogen, and the ethenyl group at C-3 position, to which heteroarylthio group is attached, may be present in the configuration of cis or trans.

The present invention also relates to a process for preparing the compound of formula (1), and to an antibacterial composition containing the compound of formula (1) as an active ingredient.

### BACKGROUND ART

Cephalosporin-based antibiotics have been widely used for treatment of infectious diseases caused by pathogenic bacteria in human and animals. They are particularly useful for treatment of diseases caused by bacteria resistant to other antibiotics such as penicillin compounds and for treatment of penicillin-hypersensitive patients. In most cases for treating such infectious diseases, it is preferred to use antibiotics showing an antimicrobial activity against both of gram-positive and gram-negative microorganisms. It has been very well known that such antimicrobial activity of cephalosporin antibiotics is highly influenced by the kind of substituents at 3- or 7-position of cephem ring. Therefore, according to the attempt to develop an antibiotic agent showing a potent antimicrobial activity against broad strains of gram-positive and gram-negative bacteria, numerous cephalosporin antibiotics having various substituents at 3- or 7-position have been developed heretofore.

Recently, resistance strains of gram-positive microorganisms, particularly methicillin-resistant Staphylococcus aureus (MRSA) have been recognized as the cause of serious hospital infection and therefore, many attempts have been made to introduce thiovinyl group into C-3 position to develop cephalosporin compounds showing a potent activity against MRSA.

For example, U.S. Pat. No. 4,719,206 discloses cephalosporin derivatives represented by the following formula (2): 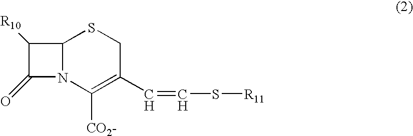
in which
R₁₀ and R₁₁ represent amino or acylamino, or
R₁₁ represents 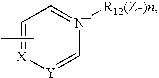
wherein
R₁₂ represents organic group,
X represents CH or N,
Z represents acid,
Y represents CH or N, and n represents 0, or
Y represents N⁺—R₁₂, wherein R₁₂ represents organic group, and n represents 1, or
Y represents -A-N⁺(R₁₃)₃, wherein R₁₃ represents lower alkyl, and
A represents lower alkylene.

Another example is the following cephalosporin derivatives of formula (3), which are disclosed in Japanese Patent No. 733,777: 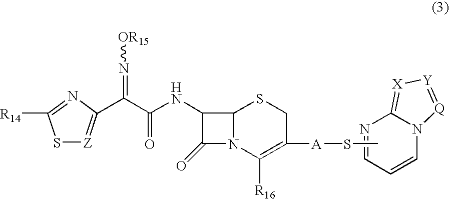
in which
R₁₄ represents amino group or protected amino,
R₁₅ represents hydrogen or organic group,
R₁₆ represents —COO—, carboxy group, or protected carboxy group,
-A- represents —CH₂— or —CH═CH—,
X, Y and Z represent CH or N,
Q represents CH, N, or N⁺—R₁₇, wherein R₁₇ represents lower alkyl.

Further, JP10,182,655, JP 5,345,787, JP 6,135,972 and U.S. Pat. No. 4,622,393 describe 3-thiovinyl cephalosporin derivatives having an antibacterial activity.

The above-mentioned prior inventions are similar to the present invention in the aspect that a double bond is introduced into C-3 position, but are different from the present invention in that the substituents attached to the double bond at C-3 position are quite different.

### DISCLOSURE OF INVENTION

Thus, the present inventors have conducted extensive and intensive researches to develop cephalosporin compounds showing broad antibacterial activity against gram-positive microorganisms including MRSA. As a result, we have identified that a certain thiovinyl cephalosporin compound having optionally substituted pyrimidinyl or heterobicyclo group at C-3 position meets the above requirement, and then completed the present invention.

### BEST MODE FOR CARRYING OUT THE INVENTION

The purpose of the present invention is to provide a novel cephalosporin compound of the following formula (1), 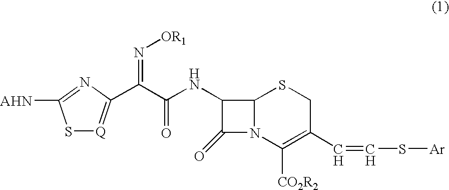
or pharmaceutically acceptable non-toxic salt, physiologically hydrolysable ester, hydrate, solvate or isomer thereof, in which
A represents hydrogen or amino-protecting group,
R₁ represents hydrogen, or represents C₁₋₆ alkyl, C₃₋₄ alkynyl, C₃₋₆ cycloalkyl or C₃₋₆ cycloalkyl-methyl, each of which may comprise one to three atoms selected from the group consisting of oxygen and halogen,
R₂ represents hydrogen or carboxyl-protecting group,
Ar represents 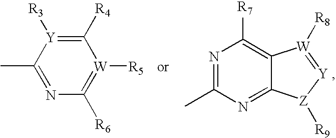
wherein
R₃, R₄, R₅ and R₇ independently of one another represent hydrogen; hydroxyl; C₁₋₆ alkyl; amino which is unsubstituted or substituted by C₁₋₆ alkyl; C₁₋₆ hydroxyalkyl; or C₁₋₆ alkylthio,
R₆ represents hydrogen; hydroxyl; amino which is unsubstituted or substituted by C₁₋₆ alkyl; C₁₋₆ alkyl; or 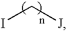
wherein
I represents S, NH, CH₂, or O,
n represents 0, 1, 2, 3, or 4,
J represents amino which is unsubstituted or substituted by C₁₋₆ alkyl; hydroxy; or C₁₋₆ alkoxy,
R₈ and R₉ independently of one another represent hydrogen; C₁₋₆ alkyl; C₁₋₆ alkylamino; hydroxy; or C₁₋₆ alkoxy,
W and Y independently of one another represent N or C, provided that R₃, R₅, R₈ do not exist when W or Y represents N,
Z represents CH or N,
Q represents CH, C—G, or N, wherein G represents halogen, and
the ethenyl group at C-3 position, to which heteroarylthio group is attached, may be present in the configuration of cis or trans.

The compound of formula (1) according to the present invention can be administered in the form of an injectable formulation or an oral formulation depending on the purpose of its use.

The compound of formula (1) according to the present invention includes in its scope syn-isomer or mixtures of syn- and anti-isomers that comprise syn-isomer in an amount of more than 90% based on the configuration of the imino group at 7-position of the cephem ring. Hydrates and solvates of the compound of formula (1) are also included in the scope of the present invention. Further, the aminothiazole group of the compound of formula (1) may form a tautomer with the iminothiazoline group as depicted below:

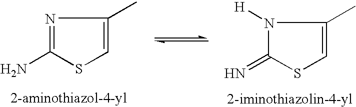

Again, the aminothiadiazole group when Q is N may form a tautomer with the iminothiadiazoline group as depicted below:

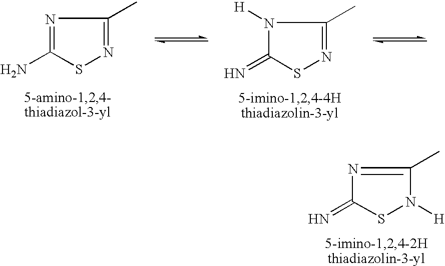

Therefore, such tautomers are also included in the scope of the present invention.

The thiovinyl group as a part of the C-3 substituent may exist in trans- or cis-isomeric form depending on the geometric configuration around the double bond as depicted below:

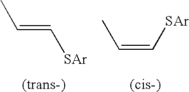

The present invention also includes the respective geometric isomers and mixtures thereof in its scope.

Pharmaceutically acceptable non-toxic salts of the compound of formula (1) include salts with inorganic acids such as hydrochloric acid, hydrobromic acid, phosphoric acid, sulfuric acid, etc., salts with organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid, malic acid, etc., or with methanesulfonic acid or para-toluenesulfonic acid, and salts with other acids which have been well-known and widely used in the technical field of penicillins and cephalosporins. These acid addition salts can be prepared according to any of the conventional methods. Further, the compound of formula (1) can also form a non-toxic salt with a base. The base that can be used for this purpose includes inorganic bases such as alkaline metal hydroxides (e.g. sodium hydroxide, potassium hydroxide, etc.), alkaline metal bicarbonates (e.g. sodium bicarbonate, potassium bicarbonate, etc.), alkaline metal carbonates (e.g. sodium carbonate, potassium carbonate, calcium carbonate, etc.), etc., and organic bases such as amino acids.

Examples of physiologically hydrolysable esters of the compound of formula (1) include indanyl, phthalidyl, methoxymethyl, pivaloyloxymethyl, glycyloxymethyl, phenylglycyloxymethyl, 5-methyl-2-oxo-1,3-dioxolen-4-ylmethyl esters or other physiologically hydrolysable esters which have been well-known and widely used in the field of penicillins and cephalosporins. These esters can be prepared according to any of the known conventional methods.

In the present invention, the preferred compounds are those wherein
G represents halogen selected from a group consisting of Cl and F,
R₁ represents hydrogen, methyl, or cyclopentyl, and
R₃, R₄, R₅ and R₇ independently of one another represent hydrogen, hydroxyl, or amino.

Typical examples of the compound of formula (1) according to the present invention include the following:
I-1: (6R,7R)-7-{[2-(2-amino-5-chloro-1,3-thiazol-4-yl)-2-(hydroxyimino)acetyl]amino}-3-{(E)-2-[(6-amino-2-hydroxy-4-pyrimidinyl)sulfanyl]ethenyl}-8-oxo-5-thia-1-azabicyclo[4.2.0]oct-2-ene-2-carboxylic acid,
I-2: (6R,7R)-7-{[2-(2-amino-5-chloro-1,3-thiazol-4-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-3: (6R,7R)-7-{[2-(2-amino-5-chloro-1,3-thiazol-4-yl)-2-(hydroxyimino)acetyl]amino}-3-{(E)-2-[(2-amino-6-hydroxy-4-pyrimidinyl)sulfanyl]ethenyl}-8-oxo-5-thia-1-azabicyclo[4.2.0]oct-2-ene-2-carboxylic acid,
I-4: (6R,7R)-7-{[2-(2-amino-5-chloro-1,3-thiazol-4-yl)-2-(hydroxyimino)acetyl]amino}-3-{(E)-2-({2-[(2-aminoethyl)sulfanyl]-4-pyrimidinyl}sulfanyl)ethenyl}-8-oxo-5-thia-1-azabicyclo[4.2.0]oct-2-ene-2-carboxylic acid,
I-5: (6R,7R)-7-{[2-(2-amino-5-chloro-1,3-thiazol-4-yl)-2-(hydroxyimino)acetyl]amino}-(E)-2-({2-[(2-aminoethyl)sulfanyl]-6-methyl-4-pyrimidinyl}sulfanyl)ethenyl)-8-oxo-5-thia-1-azabicyclo[4.2.0]oct-2-ene-2-carboxylic acid,
I-6: (6R,7R)-7-{[2-(2-amino-5-chloro-1,3-thiazol-4-yl)-2-(hydroxyimino)acetyl]amino}-3-{(E)-2-[(4-amino-1H-pyrazolo[3,4-d]pyrimidin-6-yl)sulfanyl]ethenyl}-8-oxo-5-thia-1-azabicyclo[4.2.0]oct-2-ene-2-carboxylic acid,
I-7: (6R,7R)-3-[(E)-2-({6-amino-2-[(2-aminoethyl)sulfanyl]-4-pyrimidinyl}sulfanyl) ethenyl]-7-{[2-(2-amino-5-chloro-1,3-thiazol-4-yl)-2-(hydroxyimino)acetyl]amino}-8-oxo-5-thia-1-azabicyclo[4.2.0]oct-2-ene-2-carboxylic acid,
I-8: (6R,7R)-7-{[2-(2-amino-1,3-thiazol-4-yl)-2-(methoxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-9: (6R,7R)-7-{[2-(2-amino-1,3-thiazol-4-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-10: (6R,7R)-7-({2-(2-amino-5-chloro-1,3-thiazol-4-yl)-2-[(cyclopentyloxy)imino] acetyl}amino)-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-11: (6R,7R)-3-{(E)-2-[(2-amino-6-hydroxy-4-pyrimidinyl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-12: (6R,7R)-3-{(E)-2-[(6-amino-2-hydroxy-4-pyrimidinyl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-13: (6R,7R)-7-{[2-(5-amino-1,2,4-thiadiazol-3-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-14: (6R,7R)-3-[(E)-2-({6-amino-2-[(2-aminoethyl)sulfanyl]-4-pyrimidinyl}sulfanyl) ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-15: (6R,7R)-3-[(E)-2-({2-[(2-aminoethyl)sulfanyl]-6-methyl-4-pyrimidinyl}sulfanyl) ethenyl]-7-{[2-(5-amino-1,2,4-thiadiazol-3-yl)-2-(hydroxyiminoacetyl)amino]-8-oxo-5-thia-1-azabicyclo[4.2.0]oct-2-ene-2-carboxylic acid,
I-16: (6R,7R)-3-[(E)-2-({2-[(2-aminoethyl)sulfanyl]-4-pyrimidinyl}sulfanyl)ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid, and
I-17: (6R,7R)-3-{(E)-2-[(4-amino-1H-pyrazolo[3,4-d]pyrimidin-6-yl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid.

According to the present invention, the compound of formula (1): 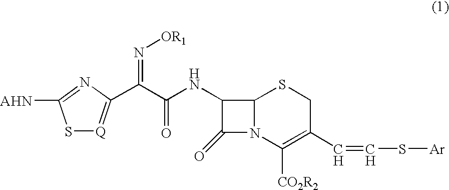
wherein A, R₁, R₂, Ar and Q are as defined above, and the double bond at C-3 position may be present in the configuration of cis or trans, and pharmaceutically acceptable non-toxic salt, physiologically hydrolysable ester, hydrate, solvate or isomer thereof can be prepared by a process which comprises reacting a compound of the following formula (4): 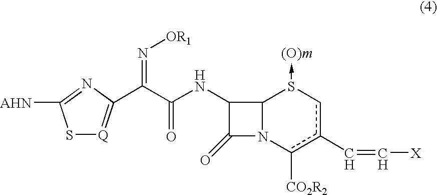
wherein A, R₁, R₂ and Q are as defined above, X represents halogen or sulfonyloxy (e.g. para-toluenesulfonyloxy, methanesulfonyloxy or trifluoromethylsulfonyloxy), m represents 0 or 1, and the double bond at C-3 position may be present in the configuration of cis or trans, with a compound of the following formula (5):
HS—Ar (5)
wherein Ar is as defined above, in a solvent to produce a compound of the following formula (1a): 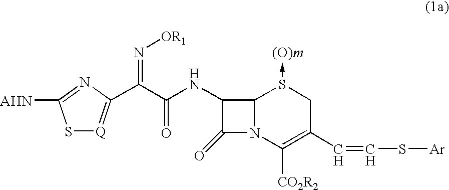
wherein A, R₁, R₂, Ar, Q, and m are as defined above, and the double bond at C-3 position may be present in the configuration of cis or trans, and if necessary, removing the amino-protecting group or acid-protecting group before or after the reaction, or reducing S→ oxide of the compound of formula (1a) wherein m is 1.

In the above formulae, the amino-protecting group A means the conventional amino-protecting groups including acyl, substituted or unsubstituted aryl(lower)alkyl (e.g. benzyl, diphenylmethyl, triphenylmethyl, 4-methoxybenzyl, etc.), halo(lower)alkyl (e.g. trichloromethyl, trichloroethyl, etc.), tetrahydropyranyl, substituted phenylthio, substituted alkylidene, substituted arylalkylidene, substituted cycloalkylidene, etc. The acyl group appropriate for the amino-protecting group may be aliphatic or aromatic acyl group, or an acyl group having a heterocycle. As the acyl group, C₁₋₅ lower alkanoyl (e.g. formyl, acetyl, etc.), C₂₋₆ alkoxycarbonyl (e.g. methoxycarbonyl, ethoxycarbonyl, etc.), lower alkanesulfonyl (e.g. methylsulfonyl, ethylsulfonyl, etc.), aryl(lower)alkoxycarbonyl (e.g. benzyloxycarbonyl, etc.), etc. can be mentioned. The acyl as mentioned above may have suitable substituents such as 1-3 halogens, hydroxy, cyano, nitro, etc. In addition, the reaction product resulting from a reaction of silane, boron, or phosphorous compound with amino group can be the amino-protecting group.

The carboxy-protecting group R² is preferably the group that can be readily removed under mild conditions. Typical examples thereof include (lower)alkyl ester (e.g. methyl ester, t-butyl ester, etc.), (lower)alkenyl ester (e.g. vinyl ester, allyl ester, etc.), (lower)alkylthio(lower)alkyl ester (e.g. methylthiomethyl ester, etc.), (lower)alkoxy(lower) alkyl ester (e.g. methyloxymethyl ester, etc.), halo(lower)alkyl ester (e.g. 2,2,2-trichloro-ethyl ester, etc.), substituted or unsubstituted arylalkyl ester (e.g. benzyl ester, p-nitrobenzyl ester, p-methoxybenzyl ester, etc.), or silyl ester, etc.

These amino-protecting groups or carboxy-protecting groups can be readily removed under mild conditions such as hydrolysis, reduction, etc. to generate a free amino- or carboxy group, and appropriately selected depending on the chemical properties of the compound of formula (1).

The leaving group X represents halogen or sulfonyloxy group (e.g. para-toluenesulfonyloxy, methanesulfonyloxy, and trifluoromethylsulfonyloxy).

The dotted line in the formulae in the present specification means, for example, each of the following formulae (6a) and (6b), or their mixture. 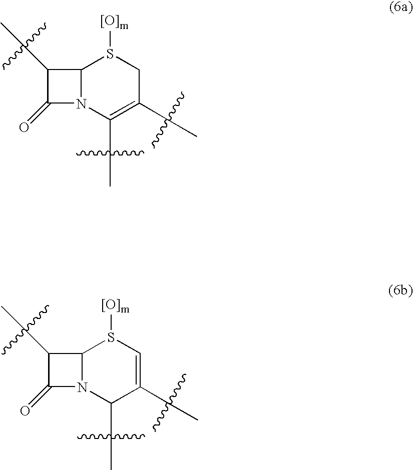
in which
m is as defined above.

The process for preparing the compound of formula (1) by introducing the compound of formula (5) into C-3 position of the compound of formula (4) may be carried out using an organic solvent. Suitable solvent for this purpose includes lower alkyl nitrites such as acetonitrile, propionitrile, etc., halogenated lower alkanes such as chloromethane, dichloromethane, chloroform, etc., ethers such as tetrahydrofuran, dioxane, ethylether etc., amides such as dimethylformamide, etc., esters such as ethyl acetate, etc., ketones such as acetone, etc., hydrocarbons such as benzene, etc., alcohols such as methanol, ethanol, etc., sulfoxides such as dimethylsulfoxide, etc., or the mixtures thereof.

The reaction temperature is generally in the range of −10 to 80° C., preferably in the range of 20 to 40° C. The compound of formula (5) is used in an amount of 0.5 to 2 equivalent weights, preferably 1.0 to 1.1 equivalent weights with respect to the compound of formula (4).

In the above process for preparing the compound of formula (1), the amino-protecting group or acid-protecting group present in the compound of formula (4) can be removed by any of the conventional methods widely known in the field of cephalosporins. That is, the protecting groups can be removed by hydrolysis or reduction. When an amido group is included in the protecting group, it is preferable to hydrolyze after amino-halogenation and amino-etherification are performed. Acid hydrolysis is useful for removing tri(di)phenylmethyl group or alkoxycarbonyl group and is carried out using an organic acid such as formic acid, trifluoroacetic acid, p-toluenesulfonic acid, etc., or an inorganic acid such as hydrochloric acid, etc.

The resulting product from the above process can be treated with various methods such as recrystallization, electrophoresis, silica gel column chromatography or ion exchange chromatography to separate and purify the desired compound of formula (1).

The compound of formula (4), an intermediate compound in the present invention, can be prepared as explained below. That is, the compound of formula (4) can be prepared by activating a compound of the following formula (7): 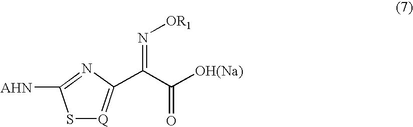
wherein A, R₁, and Q are as defined above, or salt thereof with an acylating agent, reacting the activated compound of formula (7) with a compound of the following formula (8): 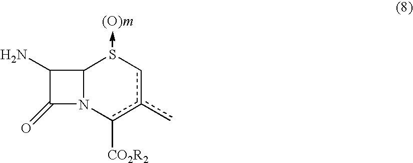
wherein R₂ and m are as defined above, to produce a compound of the following formula (9): 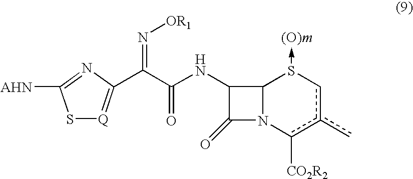
wherein A, R₁, R₂, Q and m are as defined above, and introducing a vinyl group into C-3 position of the compound of formula (9).

The dotted line in the compound of formula (8) means that the compound of formula (8) exists in the configuration of each of the following formula (8a), (8b), or (8c), or their mixture: 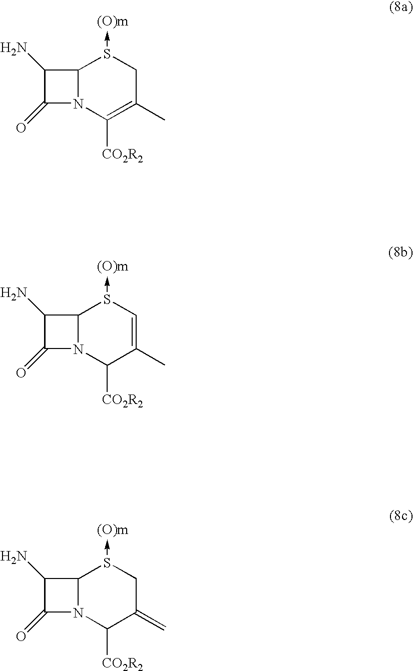
in which, R₂ and m are as defined above.

In preparing the compound of formula (9), the acylated derivative as the activated form of the compound of formula (7) includes acid chlorides, acid anhydrides, mixed acid anhydrides (preferably, acid anhydrides formed with methylchloroformate, mesitylene sulfonyl chloride, p-toluenesulfonyl chloride or chlorophosphate) or activated esters (preferably, esters formed from the reaction with N-hydroxybenzotriazole in the presence of a condensing agent such as dicyclohexylcarbodiimide), etc.

The reaction of the compound of formula (7) with the compound of formula (8) can be carried out in the presence of a condensing agent such as dicyclohexylcarbodiimide or carbonyldiimidazole, and the compound of formula (7) can also be applied to this reaction in a free acid form. This reaction is well practiced generally in the presence of an organic base, preferably a tertiary amine such as triethylamine, dimethylaniline, pyridine, etc., or an inorganic base such as sodium bicarbonate, sodium carbonate, etc. The solvent which can be used in this reaction includes halogenated hydrocarbons such as methylene chloride, chloroform, etc., tetrahydrofuran, acetonitrile, dimethylformamide or dimethylacetamide. The mixed solvent comprising two or more solvents selected from the above can be used. The reaction can also be carried out in an aqueous solution. The reaction temperature is in the range of −50 to 50° C., preferably in the range of −30 to 20° C. The compound of formula (7) can be used in an equimolar amount or a slightly excessive amount, i.e. in an amount of 1.05 to 1.2 equivalent weights, with respect to the compound of formula (8).

In order to synthesize the compound of formula (4) from the compound formula (9), it is required to introduce a vinyl group into C-3 position. For this purpose, the compound of formula (9) can react with a bis(dialkylamino)methane of the following formula (10): 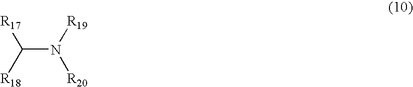
wherein R₁₇ represents OR₂₁ (wherein R₂₁ represents lower alkyl or phenyl), R₁₈ represents OR₂₁ or NR₁₉R₂₀, and R₁₉ and R₂₀ independently of one another represent lower alkyl or phenyl, to produce an enamine compound of the following formula (11): 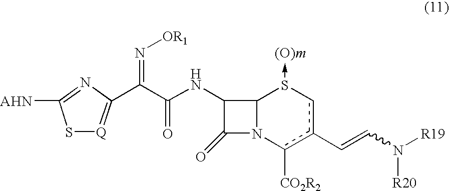
in which A, R₁, R₂, Q, m, R₁₉, and R₂₀ are as defined above, and the double bond at C-3 position may be present in the configuration of cis or trans.

The solvents which can be used for preparing the compound of formula (11) includes dimethylformamide, hexamethylphosphorotriamide, dimethylacetamide, acetonitrile, ethyl acetate, dioxane, halogenated hydrocarbons, etc. The reaction temperature is in the range of 20 to 100° C., preferably in the range of 40 to 80° C. The compound of formula (10) can be used in an amount of 1 to 5 equivalent weights with respect to the compound of formula (9).

The compound of formula (11) may be acid-hydrolyzed to produce a compound of the following formula (12): 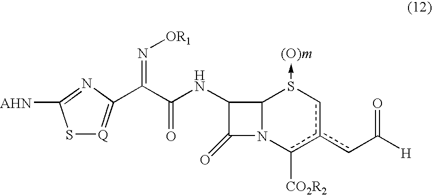
in which, A, R₁, R₂, Q and m are as defined above.

The acid which can be used for preparing the compound of formula (12) includes organic acid (formic acid or acetic acid) or mineral acid (hydrochloric acid or sulfuric acid), and the solvent includes halogenated hydrocarbons, ethyl acetate, tetrahydrofuran, acetonitrile, dimethylformamide, alcohols, etc. The hydrolysis reaction may also be carried out using only the acid without any solvent. The reaction temperature is in the range of −20 to 100° C., preferably in the range of 20 to 30° C.

The aldehyde group at C-3 position of the compound of formula (12) should be converted into a vinyloxysulfonyl group in order to prepare the compound of formula (4). This process can be carried out in accordance with any of the conventional methods. That is, the compound of formula (12) may react with an activated form of sulfonic acid (sulfonic anhydride or sulfonyl chloride) in the presence of a base to produce the compound of formula (4). The base in this reaction includes any organic base, preferably typical tertiary amines such as triethylamine, dimethylaniline, pyridine, etc. The specific examples of the activated form of sulfonic acid include para-toluenesulfonyl chloride, methanesulfonyl chloride, methanesulfonic anhydride, trifluoromethanesulfonic anhydride, etc. The activated sulfonic acid is usually used in an amount of 1 to 3 equivalent weights with respect to the compound of formula (12). As the solvent, halogenated hydrocarbons such as methylene chloride, chloroform, etc., tetrahydrofuran, acetonitrile, dimethylformamide, dimethylacetamide, etc., or their mixtures can be used. The reaction temperature is suitably in the range of −78 to 0° C.

An alternative process for preparing the compound of formula (1) according to the present invention is explained below. The compound of formula (1) can be prepared by a process which comprises subjecting a compound of the following formula (13): 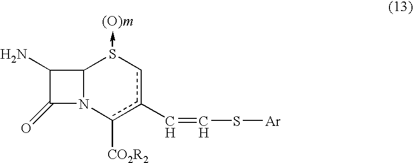
wherein Ar, R₂ and m are as defined above, and the double bond at C-3 position may be present in the configuration of cis or trans, to an amide-bond forming reaction with a compound of the following formula (7): 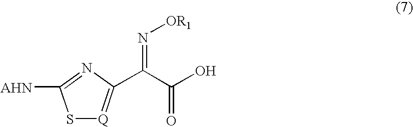
wherein A, R₁ and Q are as defined above, or a derivative thereof activated at its carboxy group in a solvent to produce a compound of the following formula (1a): 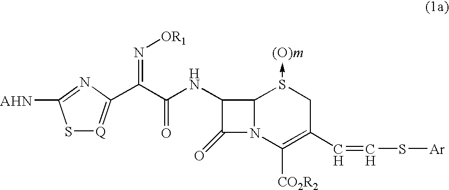
wherein A, R₁, R₂, Ar, Q, and m are as defined above, and the double bond at C-3 position may be present in the configuration of cis or trans, and if necessary, removing the amino-protecting group or acid-protecting group before or after the reaction, or reducing S oxide of the compound of formula (1a) wherein m is 1.

In the above process for preparing the compound of formula (1), the acylated derivative as the activated form of the compound of formula (7) includes acid chlorides, acid anhydrides, mixed acid anhydrides (preferably, acid anhydrides formed with methylchloroformate, mesitylene sulfonyl chloride, p-toluenesulfonyl chloride or chlorophosphate) or activated esters (preferably, esters formed from the reaction with N-hydroxybenzotriazole in the presence of a condensing agent such as dicyclohexyl-carbodiimide), etc.

The reaction of the compound of formula (7) with the compound of formula (13) can be carried out in the presence of a condensing agent such as dicyclohexylcarbodiimide or carbonyldiimidazole, and the compound of formula (7) can also be applied to this reaction in a free acid form. This reaction is well practiced generally in the presence of an organic base, preferably a tertiary amine such as triethylamine, dimethylaniline, pyridine, etc., or an inorganic base such as sodium bicarbonate, sodium carbonate, etc. The solvent which can be used in this reaction includes halogenated hydrocarbons such as methylene chloride, chloroform, etc., tetrahydrofuran, acetonitrile, dimethylformamide or dimethylacetamide. The mixed solvent comprising two or more solvents selected from the above can be used. The reaction can also be carried out in an aqueous solution. The reaction temperature is in the range of −50 to 50° C., preferably in the range of −30 to 20° C. The compound of formula (7) can be used in an equimolar amount or a slightly excessive amount, i.e. in an amount of 1.05 to 1.2 equivalent weights, with respect to the compound of formula (13).

In the above process for preparing the compound of formula (1), the amino-protecting group or acid-protecting group can be removed by any of the conventional methods widely known in the field of cephalosporins. That is, the protecting groups can be removed by hydrolysis or reduction. When an amido group is included in the protecting group, it is preferable to hydrolyze after amino-halogenation and amino-etherification are performed. Acid hydrolysis is useful for removing tri(di)phenylmethyl group or alkoxycarbonyl group and is carried out using an organic acid such as formic acid, trifluoroacetic acid, p-toluenesulfonic acid, etc., or an inorganic acid such as hydrochloric acid, etc.

The resulting product from the above process can be treated with various methods such as recrystallization, electrophoresis, silica gel column chromatography or ion exchange chromatography to separate and purify the desired compound of formula (1).

The intermediate compound of formula (13) can be prepared by removing the amino-protecting group from a compound of the following formula (14): 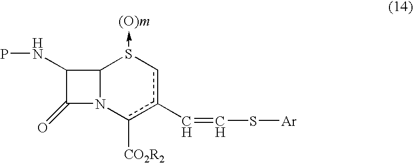
wherein R₂, m and Ar are as defined above, P represents amino-protecting group, and the double bond at C-3 position may be present in the configuration of cis or trans, using an organic acid or an inorganic acid.

In the above formulae, the amino-protecting group P means the conventional amino-protecting groups including acyl, substituted or unsubstituted aryl(lower)alkyl (e.g. benzyl, diphenylmethyl, triphenylmethyl, 4-methoxybenzyl, etc.), halo(lower)alkyl (e.g. trichloromethyl, trichloroethyl, etc.), tetrahydropyranyl, substituted phenylthio, substituted alkylidene, substituted arylalkylidene, substituted cycloalkylidene, etc. The acyl group appropriate for the amino-protecting group may be aliphatic or aromatic acyl group, or an acyl group having a heterocycle. As the acyl group, C₁₋₅ lower alkanoyl (e.g. formyl, acetyl, etc.), C₂₋₆ alkoxycarbonyl (e.g. methoxycarbonyl, ethoxycarbonyl, etc.), lower alkanesulfonyl (e.g. methylsulfonyl, ethylsulfonyl, etc.), aryl(lower)alkoxycarbonyl (e.g. benzyloxycarbonyl, etc.), etc. can be mentioned. The acyl as mentioned above may have suitable substituents such as 1-3 halogens, hydroxy, cyano, nitro, etc. In addition, the reaction product resulting from a reaction of silane, boron, or phosphorous compound with amino group can be the amino-protecting group.

The acid which can be used for preparing the compound of formula (13) includes organic acid (trifluoromethanesulfonic acid, trifluoroacetic acid, formic acid or acetic acid) or mineral acid (hydrochloric acid or sulfuric acid), and the solvent includes halogenated hydrocarbons, ethyl acetate, tetrahydrofuran, acetonitrile, dimethylformamide, alcohols, etc. The hydrolysis reaction may also be carried out using only the acid without any solvent. The reaction temperature is in the range of −50 to 50° C., preferably in the range of 0 to 15° C.

The compound of formula (14) can be prepared by replacing the X group attached to the vinyl group at C-3 position of a compound of the following formula (15): 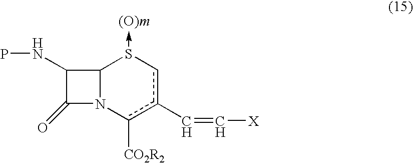
wherein P, m, R₂ and X are as defined above, and the double bond at C-3 position may be present in the configuration of cis or trans, with the compound of formula (5).

The solvent which can be used includes lower alkyl nitriles such as acetonitrile, propionitrile, etc., halogenated lower alkanes such as chloromethane, dichloromethane, chloroform, etc., ethers such as tetrahydrofuran, dioxane, ethylether etc., amides such as dimethylformamide, etc., esters such as ethyl acetate, etc., ketones such as acetone, etc., hydrocarbons such as benzene, etc., alcohols such as methanol, ethanol, etc., sulfoxides such as dimethylsulfoxide, etc., or the mixtures thereof. The leaving group X represents chlorine, fluorine, iodine, or sulfonyloxy group (e.g. para-toluenesulfonyloxy, methane-sulfonyloxy, or trifluoromethylsulfonyloxy). The reaction temperature is generally in the range of −10 to 80° C., preferably in the range of 20 to 40° C. The compound of formula (5) is used in an amount of 0.5 to 2 equivalent weights, preferably 1.0 to 1.1 equivalent weights with respect to the compound of formula (15).

The compound of formula (15) wherein X represents chlorine can be prepared by referring to European Patent No. 154354 or No. 53538. Further, in order to prepare the compound of formula (15) wherein X represents sulfonyloxy, an aldehyde group at C-3 position of a compound of the following formula (16): 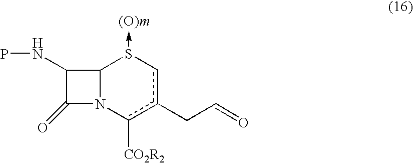
wherein P, R₂ and m are as defined above, and the double bond at C-3 position may be present in the configuration of cis or trans, should be converted into a vinyloxysulfonyl group. This process can be carried out according to any of the conventional methods as explained below.

The compound of formula (16) may react with an activated form of sulfonic acid (sulfonic anhydride or sulfonyl chloride) in the presence of a base to produce the compound of formula (15). The base in this reaction includes any organic base, preferably typical tertiary amines such as triethylamine, dimethylaniline, pyridine, etc. The specific examples of the activated form of sulfonic acid include para-toluenesulfonyl chloride, methanesulfonyl chloride, methanesulfonic anhydride, trifluoromethanesulfonic anhydride, etc. The activated sulfonic acid is usually used in an amount of 1 to 3 equivalent weights with respect to the compound of formula (16). As the solvent, halogenated hydrocarbons such as methylene chloride, chloroform, etc., tetrahydrofuran, acetonitrile, dimethylformamide, dimethylacetamide, etc. can be used. The reaction temperature is suitably in the range of −78 to 0° C.

The compound of formula (16) having various amino-protecting groups can be prepared from the starting compound of 7-amino-deacetyloxycephalosporanic acid by referring to U.S. Pat. No. 4,622,393.

The compound of formula (1) according to the present invention can be administered in the form of an injectable formulation or an oral formulation depending on the purpose of its use.

The compound of formula (1) according to the present invention can be formulated using known pharmaceutically acceptable carriers and excipients according to the known method to prepare a unit dosage form or to be introduced into a multi-dosage container. The formulations can be in the form of a solution, suspension or emulsion in an oil or aqueous medium and can contain conventional dispersant, suspending agent or stabilizing agent. In addition, the formulation can also be in the form of a ready-to-use dry powder which can be used by dissolving with a sterile, pyrogen-free water before its use. The compound of formula (1) can also be formulated in the form of a suppository by using conventional suppository bases such as cocoa butter or other glycerides. Solid dosage form for oral administration includes capsules, tablets, pills, powders and granules, with capsules and tablets being particularly advantageous. For the tablets and pills, it is preferred to provide an enteric coating. Solid dosage form can be prepared by mixing the active compound of formula (1) according to the present invention with one or more inert diluents such as sucrose, lactose, starch, etc., and carriers including lubricants such as magnesium stearate, disintegrating agents, binders, etc.

If necessary, the compound of formula (1) according to the present invention can be administered in combination with other antibacterial agent such as penicillins or cephalosporins.

In formulating the compound of formula (1) according to the present invention into the unit dosage form, it is preferred that the unit dosage form contains the active ingredient of formula (1) in an amount of about 50 to 1,500 mg. The dosage of the compound of formula (1) is suitably selected under the physician's prescription depending on various factors including weight and age of patient, particular conditions and severity of diseases to be treated, etc. However, the daily dosage for the treatment of adult man generally corresponds to about 500 to 5,000 mg depending on the frequency and intensity of administration. For intramuscular or intravenous injection to adult man, a total daily dosage in the range of about 150 to 3,000 mg is generally sufficient. However, in case of infections caused by some pathogenic strains, it may be required to increase the daily dosage.

The compound of formula (1) and its non-toxic salt (preferably salts with alkali metals, alkaline earth metals, inorganic acids, organic acids and amino acids) according to the present invention exhibit a potent antimicrobial activity and a broad antibacterial spectrum against broad pathogenic microorganisms including various gram-positive and gram-negative strains and therefore, are very useful for the prevention and treatment of diseases caused by bacterial infection in animals including human being.

The present invention will be more specifically illustrated by the following preparations and examples. However, it should be understood that they do not intend to limit the present invention in any manner.

Preparation 1

### Synthesis of benzhydryl (6R,7R)-7-({2-{2-[(t-butoxycarbonyl)amino]-5-chloro-1,3-thiazol-4-yl}-2-[(trityloxy)imino]acetyl}amino)-3-methyl-8-oxo-5-thia-1-azabicyclo[4.2.0]oct-2-ene-2-carboxylate

60 g (0.16 mol) of Benzhydryl 7-amino-3-methyl-8-oxo-5-thia-1-azabicyclo[4.2.0] oct-2-ene-2-carboxylate was dissolved in 750 ml of distilled dichloromethane, and 97.84 g (0.17 mol) of 2-{2-[(t-butoxycarbonyl)amino]-5-chloro-1,3-thiazol-4-yl}-2-[(trityl oxy)imino]acetic acid was added thereto. The temperature of the reaction vessel was lowered to −30° C., and then each of 32.1 ml (0.39 mol) of pyridine and 19.2 ml (0.21 mol) of phosphoryloxy chloride was slowly added dropwise. The temperature of the reaction vessel was gradually raised to −10˜−5° C., during which the reaction mixture was stirred for 3 hours. The reaction mixture was diluted with excess ethyl acetate, washed with saturated ammonium chloride solution, 5% sodium bicarbonate solution and sodium chloride solution, respectively, dried over anhydrous magnesium sulfate, and filtered. The filtrate was distilled under reduced pressure, and the residue was purified by column chromatography to give 97.73 g (Yield 66.9%) of the title compound.

¹H NMR(CDCl₃, 400 MHz) δ 8.28 (1H, s), 7.49˜7.24 (25H, m), 6.94 (1H, s), 6.01˜5.97 (1H, m), 5.04 (1H, d, J=8.0 Hz), 3.38˜3.33, 3.07˜3.02 (2H, ABq, J=20.0 Hz), 2.11 (3H, s), 1.50 (9H, s)

Mass (m/e) 926 (M+H⁺)

Preparation 2

### Synthesis of benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-(2-oxoethyl)-5-thia-1-azabicyclo[4.2.0]oct-2-ene-2-carboxylate

150 ml of dimethylformamide was heated to 50° C. and 50 g (53.96 mmol) of benzhydryl (6R,7R)-7-({2-{2-[(t-butoxycarbonyl)amino]-5-chloro-1,3-thiazol-4-yl}-2-[(trityloxy)imino]acetyl}amino)-3-methyl-8-oxo-5-thia-1-azabicyclo[4.2.0]oct-2-ene-2-carboxylate was added thereto. To the reaction mixture was added dropwise 33.4 ml (161.9 mmol) of t-butoxy-bis(dimethylamino)methane, which was then stirred at 50° C. for 25 minutes. The reaction mixture was diluted with excess ethyl acetate, washed with sodium chloride solution, dried over anhydrous magnesium sulfate, and filtered. The filtrate was distilled under reduced pressure. To the residue were added 600 ml of ethyl acetate and 400 ml of 1N hydrochloric acid, and the resulting mixture was stirred at room temperature for 2 hours. The mixture was diluted again with excess ethyl acetate, washed with sodium chloride solution, dried over anhydrous magnesium sulfate, and filtered. The filtrate was distilled under reduced pressure. The residue was dissolved in a small amount of diethylether, purified from n-hexane, filtered, and dried under nitrogen atmosphere to give 47.42 g (Yield 92.1%) of the title compound.

¹H NMR(CDCl₃, 400 MHz) δ 9.56 (1H, s), 8.26 (1H, bs), 7.47˜7.23 (25H, m), 6.90 (1H, s), 6.11˜6.07 (1H, m), 5.08 (1H, d, J=4.8 Hz), 3.70˜3.66, 3.53˜3.49 (2H, ABq, J=16.0 Hz), 3.51˜3.46, 3.16˜3.11 (2K ABq, J=20.0 Hz), 1.50 (9H, s)

Mass (m/e) 954 (M+H⁺)

Preparation 3

### Synthesis of benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate

27.3 g (28.6 mmol) of benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-(2-oxoethyl)-5-thia-1-azabicyclo[4.2.0]oct-2-ene-2-carboxylate was dissolved in 87.4 ml of distilled dichloromethane. The temperature of the reaction vessel was lowered to −30˜−35° C. Then, 37.4 ml (214.6 mmol) of pyridine and 7.22 ml (42.9 mmol) of trifluoromethanesulfonic anhydride were added and the resulting mixture was stirred for 15 minutes. The reaction mixture was diluted with excess ethyl acetate, neutralized by 1N hydrochloric acid, washed with sodium chloride solution, dried over anhydrous magnesium sulfate, and then filtered. The filtrate was distilled under reduced pressure and the residue was purified by column chromatography to give 10.0 g (Yield 32.2%) of the title compound.

¹H NMR(CDCl₃, 400 MHz) δ 8.17 (1H, bs), 7.46˜7.24 (25H, m), 7.13 (1H, d, J=12.0 Hz), 7.00 (1H, s), 6.88 (1H, d, J=12.0 Hz), 6.05 (1H, m), 5.09 (1H, d, J=4.0 Hz), 3.45˜3.41, 3.23˜3.19 (2H, ABq, J=16.0 Hz), 1.50 (9H, s)

Mass (m/e) 1086 (M+H⁺)

Preparation 4

### Synthesis of benzhydryl 7-({2-(methoxyimino)-2-[2-(tritylamino)-1,3-thiazol-4-yl]acetyl}amino)-3-methyl-8-oxo-5-thia-1-azabicyclo[4.2.0]oct-2-ene-2-carboxylate

The title compound (16.6 g, Yield 78%) was obtained according to the same procedure as Preparation 1 using 2-(methoxyimino)-2-[2-(tritylamino)-1,3-thiazol-4-yl] acetic acid (12.82 g, 29 mmol).

¹H NMR(CDCl₃, 400 MHz) δ 7.46˜7.26 (25H, m), 6.92 (1H, s), 6.80 (1H, s), 6.04 (1H, d, J=4.4 Hz), 4.09 (3H, s), 3.52˜3.47, 3.23˜3.19 (2H, ABq, J=18.4 Hz), 2.13 (3H, s)

Mass (m/e) 806 (M+H⁺)

Preparation 5

### Synthesis of benzhydryl 7-({2-(methoxyimino)-2-[2-(tritylamino)-1,3-thiazol-4-yl]acetyl}amino)-8-oxo-3-(2-oxoethyl)-S-thia-1-azabicyclo[4.2.0]oct-2-ene-2-carboxylate

The title compound (5.6 g, Yield 54%) was obtained according to the same procedure as Preparation 2 using benzhydryl 7-({2-(methoxyimino)-2-[2-(tritylamino)-1,3-thiazol-4-yl]acetyl}amino)-3-methyl-8-oxo-5-thia-1-azabicyclo[4.2.0]oct-2-ene-2-carboxylate (10 g, 12.4 mmol).

¹H NMR(CDCl₃, 400 MHz) δ 9.56 (1H, s), 7.49˜7.20 (25H, m), 6.90 (1H, s), 6.82 (1H, s), 5.98˜5.91 (1H, m), 5.05 (1H, d, J=4.0 Hz), 4.09 (3H, s), 3.72˜3.68, 3.55˜3.51 (2H, ABq, J=16.0 Hz), 3.53˜3.48, 3.18˜3.13 (2H, ABq, J=20.0 Hz)

Mass (m/e) 834 (M+H⁺)

Preparation 6

### Synthesis of benzhydryl 7-({2-(methoxyimino)-2-[2-(tritylamino)-1,3-thiazol-4-yl]acetyl}amino)-8-oxo-3-((E)-2-{[(trifluoromethyl)sulfonyl]oxy}ethenyl)-5-thia-1-azabicyclo[4.2.0]oct-2-ene-2-carboxylate

The title compound (2.8 g, Yield 38.4%) was obtained according to the same procedure as Preparation 3 using benzhydryl 7-({2-(methoxyimino)-2-[2-(tritylamino)-1,3-thiazol-4-yl]acetyl}amino)-8-oxo-3-(2-oxoethyl)-5-thia-1-azabicyclo[4.2.0]oct-2-ene-2-carboxylate (5.6 g, 6.7 mmol).

Mass (m/e) 966 (M+H⁺)

Preparation 7

### Synthesis of benzhydryl 7-({2-{2-[(t-butoxycarbonyl)amino]-1,3-thiazol-4-yl}-2-[(trityloxy)imino]acetyl}amino)-3-methyl-8-oxo-5-thia-1-azabicyclo[4.2.0]oct-2-ene-2-carboxylate

The title compound (9.26 g, Yield 55%) was obtained according to the same procedure as Preparation 1 using 2-{2-[(t-butoxycarbonyl)amino]-1,3-thiazol-4-yl}-2-[(trityloxy)imino]acetic acid (10 g, 18.8 mmol).

Mass (m/e) 892 (M+H⁺)

Preparation 8

### Synthesis of benzhydryl 7-({2-{2-[(t-butoxycarbonyl)amino]-1,3-thiazol-4-yl}-2-[(trityloxy)imino]acetyl}amino)-8-oxo-3-(2-oxoethyl)-5-thia-1-azabicyclo[4.2.0]oct-2-ene-2-carboxylate

The title compound (10 g, Yield 97.0%) was obtained according to the same procedure as Preparation 2 using benzhydryl 7-({2-{2-[(t-butoxycarbonyl)amino]-1,3-thiazol-4-yl}-2-[(trityloxy)imino]acetyl}amino)-3-methyl-8-oxo-5-thia-1-azabicyclo[4.2.0]oct-2-ene-2-carboxylate (10 g, 11.2 mmol).

¹H NMR(CDCl₃, 400 MHz) δ 9.55 (1H, s), 7.47˜7.25 (25H, m), 7.05 (1H, s), 6.89 (1H, s), 6.11˜6.07 (1H, m), 5.09 (1H, d, J=4.0 Hz), 3.70˜3.66, 3.54˜3.50 (2H, ABq, J=16.0 Hz), 3.49˜3.45, 3.16˜3.12 (21K, ABq, J=16.0 Hz), 1.51 (9H, s)

Mass (m/e) 920 (M+H⁺)

Preparation 9

### Synthesis of benzhydryl 7-({2-{2-[(t-butoxycarbonyl)amino]-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate

The title compound (3.2 g, Yield 27.0%) was obtained according to the same procedure as Preparation 3 using benzhydryl 7-({2-{2-[(t-butoxycarbonyl)amino]-1,3-thiazol-4-yl}-2-[(trityloxy)imino]acetyl}amino)-8-oxo-3-(2-oxoethyl)-5-thia-1-azabicyclo [4.2.0]oct-2-ene-2-carboxylate (10 g, 10.8 mmol).

Mass (m/e) 1052 (M+H⁺)

Preparation 10

### Synthesis of benzhydryl 7-({2-{2-[(t-butoxycarbonyl)amino]-5-chloro-1,3-thiazol-4-yl}-2-[(cyclopentyloxy)imino]acetyl}amino)-3-methyl-8-oxo-5-thia-1-azabicyclo[4.2.0]oct-2-ene-2-carboxylate

The title compound (4.76 g, Yield 62%) was obtained according to the same procedure as Preparation 1 using 2-{2-[(t-butoxycarbonyl)amino]-5-chloro-1,3-thiazol-4-yl}-2-[(cyclopentyloxy)imino]acetic acid (1.53 g, 3.9 mmol).

Mass (m/e) 752 (M+H⁺)

Preparation 11

### Synthesis of benzhydryl 7-({2-{2-[(t-butoxycarbonyl)amino]-5-chloro-1,3-thiazol-4-yl}-2-[(cyclopentyloxy)imino]acetyl}amino)-8-oxo-3-(2-oxoethyl)-5-thia-1-azabicyclo[4.2.0]oct-2-ene-2-carboxylate

The title compound (2.45 g, Yield 49.0%) was obtained according to the same procedure as Preparation 2 using benzhydryl 7-({2-{2-[(t-butoxycarbonyl)amino]-5-chloro-1,3-thiazol-4-yl}-2-[(cyclopentyloxy)imino]acetyl}amino)-3-methyl-8-oxo-5-thia-1-azabicyclo[4.2.0]oct-2-ene-2-carboxylate (4.76 g, 6.3 mmol).

Mass (m/e) 780 (M+H⁺)

Preparation 12

### Synthesis of benzhydryl 7-({2-{2-[(t-butoxycarbonyl)amino]-5-chloro-1,3-thiazol-4-yl}-2-[(cyclopentyloxy)imino]acetyl}amino)-8-oxo-3-((E)-2-{[(trifluoromethyl)sulfonyl] oxy}ethenyl)-5-thia-1-azabicyclo[4.2.0]oct-2-ene-2-carboxylate

The title compound (2.5 g, Yield 73.0%) was obtained according to the same procedure as Preparation 3 using benzhydryl 7-({2-{2-[(t-butoxycarbonyl)amino]-5-chloro-1,3-thiazol-4-yl}-2-[(cyclopentyloxy)imino]acetyl}amino)-8-oxo-3-(2-oxoethyl)-5-thia-1-azabicyclo[4.2.0]oct-2-ene-2-carboxylate (2.45 g, 3.14 mmol).

Mass (m/e) 912 (M+H⁺)

Preparation 13

### Synthesis of benzhydryl 7-[(t-butoxycarbonyl)amino]-8-oxo-3-((E)-2-{[(tri-fluoromethyl)sulfonyl] oxy}ethenyl)-5-thia-1-azabicyclo[4.2.0]oct-2-ene-2-carboxylate

2.57 g (5.06 mmol) of benzhydryl 7-[(t-butoxycarbonyl)amino]-8-oxo-3-(2-oxoethyl)-5-thia-1-azabicyclo[4.2.0]oct-2-ene-2-carboxylate was dissolved in 17 ml of distilled dichloromethane. The temperature of the reaction vessel was lowered to −78° C., and 1.32 ml (7.59 mmol) of N,N-diisopropylethylamine and 1.28 ml (7.59 mmol) of trifluoromethanesulfonic anhydride were slowly added dropwise, respectively. The temperature of the reaction vessel was maintained at −78° C., during which the reaction mixture was stirred for 20 minutes. The reaction mixture was diluted with excess dichloromethane and water, washed with sodium chloride solution, dried over anhydrous magnesium sulfate, and filtered. The filtrate was distilled under reduced pressure to give 3.0 g (Yield 98.0%) of the title compound.

¹H NMR(CDCl₃, 400 MHz) δ 7.43˜7.25 (10H, m), 7.00 (1H, d, J=13.2 Hz), 6.95 (1H, d, J=13.2 Hz), 6.94 (1H, s), 5.68 (1K m), 5.23 (1H, d, J=10.0 Hz), 5.03 (1H, d, J=5.2 Hz), 3.73˜3.69, 3.61˜3.57 (2H, ABq, J=17.4 Hz), 1.49 (9H, s)

Mass (m/e) 641 (M+H⁺)

Preparation 14

### Synthesis of 7-amino-3-{(E)-2-[(2,6-diamino-4-pyrimidinyl)sulfanyl]ethenyl}-8-oxo-5-thia-1-azabicyclo[4.2.0]oct-2-ene-2-carboxylic acid

3 g (4.97 mmol) of benzhydryl 7-[(t-butoxycarbonyl)amino]-8-oxo-3-((E)-2-{[(trifluoromethyl)sulfonyl]oxy}ethenyl)-5-thia-1-azabicyclo[4.2.0]oct-2-ene-2-carboxylate was dissolved in 30 ml of dimethylformamide. To the reaction vessel was added 1.23 g (6.46 mmol) of 2,4-diamino-6-mercaptopyrimidine 1/2 sulfate, and the mixture was reacted at room temperature for 24 hours. Excess ethyl acetate was added and the reaction mixture was washed with sodium chloride solution. The organic layer was dried over anhydrous magnesium sulfate and filtered. The filtrate was distilled under reduced pressure and the residue was purified by column chromatography.

The solid 1.39 g as obtained above was deprotected using trifluoroacetic acid, anisole and triethylsilane, purified by diethylether, and then filtered. The solid thus obtained was dried under nitrogen atmosphere to give 886 mg (Yield of two steps 38.6%) of the title compound.

¹H NMR (D₂O, 400 MHz) δ 6.62 (1H, d, J=10.4 Hz), 6.35 (1H, d, J=10.4 Hz), 5.81 (1H, s), 5.07 (11, d, J=5.2 Hz), 4.93 (1H, d, J=4.8 Hz), 3.63˜3.50 (1H, m)

Mass (m/e) 367 (M+H⁺)

Preparation 15

### Synthesis of benzhydryl (6R,7R)-7-(2-{5-[(t-butoxycarbonyl)amino]-1,2,4-thiadiazol-3-yl}-2-{[(trityloxy)imino]acetyl}amino)-3-methyl-8-oxo-5-thia-1-azabicyclo[4.2.0]oct-2-ene-2-carboxylate

Benzhydryl 7-amino-3-methyl-8-oxo-5-thia-1-azabicyclo[4.2.0]oct-2-ene-2-carboxylate (5.61 g, 14.74 mmol) and 2-{5-[(t-butoxycarbonyl)amino]-1,2,4-thiadiazol-3-yl}-2-[(trityloxy)imino] acetic acid (7.83 g, 14.75 mmol) were reacted according to the same procedure as Preparation 1 to give the title compound (13.2 g, Yield 99%).

¹H NMR(CDCl₃, 500 MHz) δ 7.57.1 (25H, m), 6.92 (1H, s), 6.04˜6.02 (1H, m), 5.04 (1H, d, 5.0 Hz), 3.34 (1H, d, 18.3 Hz), 3.04 (1H, d, 17.9 Hz), 2.1 (3H, s), 1.51 (9H, s)

Mass 893 (M+H⁺)

Preparation 16

### Synthesis of benzhydryl (6R,7R)-7-(2-{5-[(t-butoxycarbonyl)amino]-1,2,4-thia diazol-3-yl}-2-{[(trityloxy)imino]acetyl}amino)-8-oxo-3-(2-oxoethyl)-5-thia-1-azabicyclo[4.2.0]oct-2-ene-2-carboxylate

Benzhydryl (6R,7R)-7-(2-{5-[(t-butoxycarbonyl)amino]-1,2,4-thiadiazol-3-yl}-2-{[(trityloxy)imino]acetyl}amino)-3-methyl-8-oxo-5-thia-1-azabicyclo[4.2.0]oct-2-ene-2-carboxylate (13.2 g, 14.78 mmol) and t-butoxy-bis(dimethylamino)methane (7.6 g, 43.6 mmol) were reacted according to the same procedure as Preparation 2 to give the title compound (11.8 g, Yield 86%).

¹NMR(CDCl₃ 400 MHz) δ 9.53 (1H, brs), 7.50˜7.01 (25K m), 6.91 (1H, s), 6.06˜6.03 (1H, m), 5.07 (1H, d, 5.2 Hz), 3.62 (1H, d, 16.4 Hz), 3.50 (1H, d, 16.4 Hz), 3.43 (1H, d, 18.4 Hz), 3.12 (1H, d, 18 Hz), 1.53 (9H, s)

Mass 922 (M+H⁺)

Preparation 17

### Synthesis of benzhydryl (6R,7R)-7-(2-{5-[(t-butoxycarbonyl)amino]-1,2,4-thia diazol-3-yl}-2-{[(trityloxy)imino]acetyl}amino)-8-oxo-3-((E)-2-{[(trifluoromethyl)sulfonyl] oxy}ethenyl)-5-thia-1-azabicyclo[4.2.0]oct-2-ene-2-carboxylate

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-(2-oxoethyl)-5-thia-1-azabicyclo[4.2.0]oct-2-ene-2-carboxylate (11.8 g, 12.8 mmol) was reacted according to the same procedure as Preparation 3 to give the title compound (4 g, Yield 29%).

¹H NMR(CDCl₃, 400 MHz) δ 7.61˜7.1 (26H, m), 7.00 (1H, s), 6.83 (1H, d, 12.4 Hz), 6.05˜5.99 (1H, m), 5.09 (1H, d, 4.4 Hz), 3.42 (1H, d, 17.6 Hz), 3.21 (1H, d, 18.0 Hz), 1.50 (9H, s)

Mass 1053 (M+H⁺)

### EXAMPLE 1

### Synthesis of (6R,7R)-7-{[2-(2-amino-5-chloro-1,3-thiazol-4-yl)-2-(hydroxy imino)acetyl]amino}-3-{(E)-2-[(6-amino-2-hydroxy-4-pyrimidinyl)sulfanyl]ethenyl}-8-oxo-5-thia-1-azabicyclo[4.2.0]oct-2-ene-2-carboxylic acid

11.33 g (10.4 mmol) of benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate was dissolved in 35 ml of dimethylformamide and 17 ml of dimethylsulfoxide. To the reaction vessel was added 3.01 g (15.7 mmol) of 4-amino-6-mercapto-2-pyrimidinol sulfate, and the mixture was reacted at room temperature for 24 hours. Excess ethyl acetate was added, and the mixture was washed with sodium chloride solution. The organic layer was dried over anhydrous magnesium sulfate and filtered. The filtrate was distilled under reduced pressure and the residue was purified by column chromatography.

The solid 7.93 g as obtained above was deprotected using trifluoroacetic acid and triethylsilane, and then purified by preparative high pressure liquid chromatography to give 780 mg (Yield of two steps 13.1%) of the title compound.

¹H NMR (D₂O, 400 MHz) δ 7.07 (1H, d, J=15.2 Hz), 6.63 (1H, d, J=15.2 Hz), 5.88 (1H, d, J=4.8 Hz), 5.86 (1H, s), 5.28 (1H, d, J=4.8 Hz), 3.80˜3.76, 3.72˜3.68 (2H, ABq, J=17.2 Hz)

Mass (m/e) 571 (M+H⁺)

### EXAMPLE 2

### Synthesis of (6R,7R)-7-{[2-(2-amino-5-chloro-1,3-thiazol-4-yl)-2-(hydroxy imino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid

Benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-₂-{[(trifluoromethyl)sulfonyl]oxy} ethenyl)-5-thia-1-azabicyclo[4.2.0]oct-2-ene-2-carboxylate (7.7 g, 7 mmol) and 2,6-diamino-4-pyrimidinthiol sulfate (2.0 g, 10.4 mmol) were reacted according to the same procedure as Example 1 to give the title compound (574 mg, Yield of two steps 14%).

¹H NMR (D₂O, 400 MHz) δ 6.84 (1H, d, J=15.6 Hz), 6.64 (1H, d, J=15.6 Hz), 5.72 (1H, d, J=4.8 Hz), 5.12 (1H, d, J=4.8 Hz), 3.66˜3.62, 3.57˜3.53 (2H, Abq, J=17.6 Hz)

Mass (m/e) 570 (M+H⁺)

### EXAMPLE 3

### Synthesis of (6R,7R)-7-{[2-(2-amino-5-chloro-1,3-thiazol-4-yl)-2-(hydroxy imino)acetyl]amino}-3-{(E)-2-[(2-amino-6-hydroxy-4-pyrimidinyl)sulfanyl]ethenyl}-8-oxo-5-thia-1-azabicyclo[4.2.0]oct-2-ene-2-carboxylic acid

Benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (10 g, 9.3 mmol) and 2-amino-6-mercapto-4-pyrimidinol sulfate (2.67 g, 13.9 mmol) were reacted according to the same procedure as Example 1 to give the title compound (1.0 g, Yield of two steps 20%).

¹H NMR (D₂O, 400 MHz) δ 7.00 (1H, d, J=15.6 Hz), 6.78 (11, d, J=15.6 Hz), 5.88 (1H, d, J=2.8 Hz), 5.75 (1H, d, J=2.0 Hz), 5.27-5.26 (1H, m), 3.82˜3.78, 3.72˜3.68 (2H, ABq, J=16.8 Hz)

Mass (m/e) 571 (M+H⁺)

### EXAMPLE 4

### Synthesis of (6R,7R)-7-{[2-(2-amino-5-chloro-1,3-thiazol-4-yl)-2-(hydroxy imino)acetyl]amino}-3-{(E)-2-({2-[(2-aminoethyl)sulfanyl]-4-pyrimidinyl}sulfanyl) ethenyl}-8-oxo-5-thia-1-azabicyclo[4.2.0]oct-2-ene-2-carboxylic acid

Benzhydryl (6R,7R)-7-({2-{2-[(1-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (2 g, 1.8 mmol) and 2-{[2-(trityl amino)ethyl]sulfanyl}-4-pyrimidinthiol (1.0 g, 2.3 mmol) were reacted according to the same procedure as Example 1 to give the title compound (25 mg, Yield of two steps 2.2%).

¹H NMR (DMSO-d₆, 500 MHz) δ 8.32 (1H, d, J=5.0 Hz), 7.37 (1H, d, J=15.6 Hz), 7.20 (1H, d, J=5.0 Hz), 6.67 (1H, d, J=15.6 Hz), 5.68 (1H, m), 5.04 (1H, d, J=5.1 Hz), 3.68˜3.64, 3.54˜3.50 (2H, ABq, J=20.0 Hz), 3.37 (2H, m), 3.19 (2H, m), 1.80 (2H, s)

Mass (m/e) 615 (M+H⁺)

### EXAMPLE 5

### Synthesis of (6R,7R)-7-{[2-(2-amino-5-chloro-1,3-thiazol-4-yl)-2-(hydroxy imino)acetyl]amino}-3-{(E)-2-({2-[(2-aminoethyl)sulfanyl]-6-methyl-4-pyrimidinyl} sulfanyl)ethenyl}-8-oxo-5-thia-1-azabicyclo[4.2.0]oct-2-ene-2-carboxylic acid

Benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (2.0 g, 1.8 mmol) and 6-methyl-2-{[2-(tritylamino)ethyl]sulfanyl}-4-pyrimidinthiol (1.1 g, 2.4 mmol) were reacted according to the same procedure as Example 1 to give the title compound (20 mg, Yield of two steps 1.7%).

¹H NMR (D₂O, 400 MHz) δ 6.78 (1H, d, J=16.0 Hz), 6.72 (1H, d, J=16.0 Hz), 6.64 (1H, s), 5.57 (1H, d, 3=4.8 Hz), 4.97 (1H, d, J=4.8 Hz), 3.48˜3.44, 3.40˜3.36 (2H, ABq, J=17.2 Hz), 3.19 (2H, t, J=6.4 Hz), 3.05˜2.96 (2H, m), 2.01 (3H, s), 1.59 (2H, s)

Mass (m/e) 629 (M+H⁺)

### EXAMPLE 6

### Synthesis of (6R,7R)-7-{[2-(2-amino-5-chloro-1,3-thiazol-4-yl)-2-(hydroxy imino)acetyl]amino}-3-{(E)-2-[(4-amino-1H-pyrazolo[3,4-d]pyrimidin-6-yl)sulfanyl] ethenyl}-8-oxo-5-thia-1-azabicyclo[4.2.0]oct-2-ene-2-carboxylic acid

Benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (2.0 g, 1.8 mmol) and 4-amino-1H-pyrazolo-[3,4-d]-pyrimidin-6-thiol (833 mg, 4.9 mmol) were reacted according to the same procedure as Example 1 to give the title compound (188 mg, Yield of two steps 17.1%).

¹H NMR (DMSO-d₆, 400 MHz) δ 13.10 (1H, s), 11.55 (1H, s), 9.21 (1H, d, J=8.0 Hz), 7.85 (1H, s), 7.16 (2H, s), 7.06 (1H, d, J=16.4 Hz), 6.80 (1H, d, J=16.4 Hz), 5.47˜5.43 (1H, m), 4.91 (1H, d, J=4.8 Hz), 3.44˜3.35 (2H, m)

Mass (m/e) 595 (M+H⁺)

### EXAMPLE 7

### Synthesis of (6R,7R)-3-[(E)-2-({6-amino-2-[(2-aminoethyl)sulfanyl]-4-pyrimidinyl}sulfanyl)ethenyl]-7-{[2-(2-amino-5-chloro-1,3-thiazol-4-yl)-2-(hydroxy imino)acetyl]amino}-8-oxo-5-thia-1-azabicyclo[4.2.0]oct-2-ene-2-carboxylic acid

Benzhydryl (6R,7R)-7-({2-{2-[(t-butoxycarbonyl)amino]-5-chloro-1,3-thiazol-4-yl}-2-[(trityloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (566 mg, 0.52 mmol) and t-butyl 6-mercapto-2-{[2-(tritylamino)ethyl]sulfanyl}-4-pyrimidinylcarbamate (471 mg, 0.86 mmol) were reacted according to the same procedure as Example 1 to give the title compound (84 mg, Yield of two steps 25.5%).

¹H NMR (DMSO-d₆, 500 MHz) δ 9.38 (1H, d, J=7.8 Hz), 7.32 (2H, s), 7.06 (1H, d, J=15.6 Hz), 6.99 (1H, s), 6.66 (1H, d, J=15.6 Hz), 6.07 (1H, s), 5.65˜5.63 (1H, m), 5.06 (1H, d, J=5.1 Hz), 3.53˜3.47 (2H, m), 3.20˜3.14 (2H, m), 3.07˜3.03 (2H, m)

Mass (m/e) 630 (M+H⁺)

### EXAMPLE 8

### Synthesis of (6R,7R)-7-{[2-(2-amino-1,3-thiazol-4-yl)-2-(methoxyimino)acetyl] amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid

Benzhydryl 7-({2-(methoxyimino)-2-[2-(tritylamino)-1,3-thiazol-4-yl]acetyl} amino)-8-oxo-3-((E)-2-{[(trifluoromethyl)sulfonyl]oxy}ethenyl)-5-thia-1-azabicyclo[4.2.0]oct-2-ene-2-carboxylate (2.8 g, 2.6 mmol) and 2,6-diamino-4-pyrimidinthiol sulfate (1.93 g, 10 mmol) were reacted according to the same procedure as Example 1 to give the title compound (200 mg, Yield of two steps 14.1%).

¹H NMR (D₂O, 400 MHz) δ 6.82 (1H, s), 6.79 (1H, d, J=15.6 Hz), 6.59 (1H, d, J=15.6 Hz), 5.74 (1H, s), 5.62 (1H, d, J=4.8 Hz), 5.07 (1H, d, J=4.8 Hz), 3.79 (3H, s), 3.61˜3.57, 3.53˜3.49 (2H, ABq, J=16.4 Hz)

Mass (m/e) 550 (M+H⁺)

### EXAMPLE 9

### Synthesis of (6R,7R)-7-{[2-(2-amino-1,3-thiazol-4-yl)-2-(hydroxyimino)acetyl] amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid

Benzhydryl 7-({2-{2-[(t-butoxycarbonyl)amino]-1,3-thiazol-4-yl}-2-[(trityloxy) imino]acetyl}amino)-8-oxo-3-((E)-2-{[(trifluoromethyl)sulfonyl]oxy}ethenyl)-5-thia 1-azabicyclo[4.2.0]oct-2-ene-2-carboxylate (3.2 g, 3.0 mmol) and 2,6-diamino-4-pyrimidinthiol sulfate (3.2 g, 16.7 mmol) were reacted according to the same procedure as Example 1 to give the title compound (240 mg, Yield of two steps 15.0%).

¹H NMR (D₂O, 500 MHz) δ 7.01 (1H, d, J=15.6 Hz), 6.99 (1H, s), 6.77 (d, J=15.6 Hz), 5.92 (1H, s), 5.86 (1H, d, J=5.0 Hz), 5.30 (1, d, J=5.0 Hz), 3.81˜3.77, 3.73˜3.69 (2K ABq, J=17.4 Hz)

Mass (m/e) 536 (M+H⁺)

### EXAMPLE 10

### Synthesis of (6R,7R)-7-({2-(2-amino-5-chloro-1,3-thiazol-4-yl)-2-[(cyclopentyl oxy)imino]acetyl}amino)-3-{(E)-2-[(2,6-diamino-4-pyrimidinyl)sulfanyl]ethenyl}-8-oxo-5-thia-1-azabicyclo[4.2.0]oct-2-ene-2-carboxylic acid

Benzhydryl 7-({2-{2-[(t-butoxycarbonyl)amino]-5-chloro-1,3-thiazol-4-yl}-2-[(cyclopentyloxy)imino]acetyl}amino)-8-oxo-3-((E)-2-{[(trifluoromethyl)sulfonyl]oxy} ethenyl)-5-thia-1-azabicyclo[4.2.0]oct-2-ene-2-carboxylate (2.5 g, 2.7 mmol) and 2,6-diamino-4-pyrimidinthiol sulfate (901 mg, 4.7 mmol) were reacted according to the same procedure as Example 1 to give the title compound (200 mg, Yield of two steps 13.0%).

¹H NMR (DMSO-d₆, 400 MHz) δ 9.45 (1H, d, J=8.4 Hz), 7.38 (2H, s), 7.21 (1H, d, J=16.0 Hz), 6.58 (1H, d, J=16.0 Hz), 6.22 (2H, s), 5.98 (2H, s), 5.64 (1H, s), 5.57˜5.54 (1H, m), 5.64 (1H, d, J=5.2), 4.67 (1H, t, J=2.8), 3.59˜3.55, 3.51˜3.47 (21L ABq, J=17.2), 1.93˜1.64 (6H, m), 1.48˜1.46 (2H, m)

Mass (m/e) 638 (M+H⁺)

### EXAMPLE 11

### Synthesis of (6R,7R)-7-{[2-(2-amino-5-chloro-1,3-thiazol-4-yl)-2-(hydroxy imino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid

340 mg (0.74 mmol) of 7-amino-3-{(E)-2-[(2,6-diamino-4-pyrimidinyl)sulfanyl] ethenyl}-8-oxo-5-thia-1-azabicyclo[4.2.0]oct-2-ene-2-carboxylic acid was dissolved in 8m of dichloromethane. 0.91 ml (3.68 mmol) of N,O-bis(trimethylsilyl)acetamide was added thereto, and the mixture was stirred at room temperature for 2 hours. The temperature of the reaction vessel was lowered to 0° C. and 0.09 ml (1.10 mmol) of pyridine and 788 mg (1.10 mmol) of t-butyl 5-chloro-4-{2-[(diethoxyphosphorothioyl)oxy]-2-oxo ethaneimidoyl}-1,3-thiazol-2-yl carbamate were added. The temperature of the reaction vessel was slowly raised to room temperature and the reaction mixture was stirred for 1 hour at room temperature. Water was added in a small amount, and the mixture was purified by diethylether and filtered. The solid thus obtained was dried under nitrogen atmosphere.

The solid as obtained above was deprotected using trifluoroacetic acid, anisole and triethylsilane, purified from diethylether, filtered, and then purified by preparative high pressure liquid chromatography to give 21 mg (Yield 4.9%) of the title compound.

¹H NMR (D₂O, 400 MHz) δ 6.84 (1H, d, J=15.6 Hz), 6.64 (1H, d, J=15.6 Hz), 5.72 (1H, d, J=4.8 Hz), 5.12 (1H, d, J=4.8 Hz), 3.66˜3.62, 3.57˜3.53 (2H, ABq, J=17.6 Hz)

Mass (m/e) 570 (M+H⁺)

### EXAMPLE 12

### Synthesis of (6R,7R)-3-{(E)-2-[(2-amino-6-hydroxy-4-pyrimidinyl)sulfanyl] ethenyl}-7-1{[(5-amino-1,2,4-thiadiazol-3-yl)-2-(hydroxyimino)acetyl]amino}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and 2-amino-6-mercapto-4-pyrimidinol sulfate (430 mg, 2.2 mmol) were reacted according to the same procedure as Example 1 to give the title compound (179 mg, Yield 0.2%).

¹H NMR (DMSO, 400 MHz) δ 9.39 (11, d, 6.6 Hz), 8.03 (2H, brs), 7.24 (11 d, 12.8 Hz), 6.94 (1H, brs), 6.53 (1H, d, 12.8 Hz), 5.65˜5.62 (1H, m), 5.45 (1×, s), 5.03 (1H, d, 4.0 Hz), 3.65 (1H, d, 13.2 Hz), 3.48 (1H, d, 13.5 Hz)

Mass 538 (M+H⁺)

### EXAMPLE 13

### Synthesis of (6R,7R)-3-{(E)-2-[(6-amino-2-hydroxy-4-pyrimidinyl)sulfanyl] ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and 4-amino-6-mercapto-2-pyrimidinol sulfate (450 mg, 2.3 mmol) were reacted according to the same procedure as Example 1 to give the title compound (158 mg, Yield 0.15%).

Mass 538 (M+H⁺)

### EXAMPLE 14

### Synthesis of (6R,7R)-7-{[2-(5-amino-1,2,4-thiadiazol-3-yl)-2-(hydroxyimino) acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-S-thia-1-azabicyclo[4.2.0]oct-2-ene-2-carboxylate (2 g, 1.9 mmol) prepared in Preparation 17 and 2,6-diamino-4-pyrimidinthiol sulfate (440 mg, 2.3 mmol) were reacted according to the same procedure as Example 1 to give the title compound (184 mg, Yield 0.2%).

Mass 537 (M+H⁺)

### EXAMPLE 15

### Synthesis of (6R,7R)-3-[(E)-2-({6-amino-2-[(2-aminoethyl)sulfanyl]-4-pyrimidinyl}sulfanyl)ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and t-butyl 6-mercapto-2-{[2-(tritylamino)ethyl]sulfanyl}-4-pyrimidinyl carbamate (1.5 g, 2.7 mmol) were reacted according to the same procedure as Example 1 to give the title compound (82 mg, Yield 7%).

Mass 597 (M+H⁺)

### EXAMPLE 16

### Synthesis of (6R,7R)-3-[(E)-2-({2-[(2-aminoethyl)sulfanyl]-6-methyl-4-pyrimidinyl}sulfanyl)ethenyl]-7-{[2-(5-amino-1,2,4-thiadiazol-3-yl)-2-(hydroxyimino acetyl)amino]-8-oxo-5-thia-1-azabicyclo[4.2.0]oct-2-ene-2-carboxylic acid

Benzhydryl (6R,7R)-7-(2-{5-[(1-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and 6-methyl-2-{[2-(tritylamino)ethyl]sulfanyl}-4-pyrimidinthiol (1.2 g, 2.7 mmol) were reacted according to the same procedure as Example 1 to give the title compound (102 mg, Yield 9%).

Mass 596 (M+H⁺)

### EXAMPLE 17

### Synthesis of (6R,7R)-3-[(E)-2-({2-[(2-aminoethyl)sulfanyl]-4-pyrimidinyl} sulfanyl)ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and 2-{[2-(tritylamino)ethyl]sulfanyl}-4-pyrimidinthiol (1.1 g, 2.5 mmol) were reacted according to the same procedure as Example 1 to give the title compound (90 mg, Yield 8%).

Mass 582 (M+H⁺)

### EXAMPLE 18

### Synthesis of (6R,7R)-3-{(E)-2-[(4-amino-1H-pyrazolo[3,4-d]pyrimidin-6-yl) sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid

Benzhydryl (6R,7R)-7-(2-{5-[(t-butoxycarbonyl)amino]-1,2,4-thiadiazol-3-yl}-2-{[(trityloxy)imino]acetyl}amino)-8-oxo-3-((E)-2-{[(trifluoromethyl)sulfonyl]oxy}ethenyl)-5-thia-1-azabicyclo[4.2.0]oct-2-ene-2-carboxylate (2 g, 1.9 mmol) prepared in Preparation 17 and 4-amino-1H-pyrazolo[3,4-d]pyrimidin-6-thiol (700 mg, 4.1 mmol) were reacted according to the same procedure as Example 1 to give the title compound (122 mg, Yield 11%).

Mass 562 (M+H⁺)

Experiment 1

Minimum Inhibitory Concentration (MIC)

The effectiveness of the compound according to the present invention was determined by obtaining Minumum Inhibitory Concentration (MIC) of the compounds prepared by the above examples (Compounds I-1˜I-6, I-10) and Vancomycin, which is the known compound having a potent activity against gram-positive strains, as the control drug against the standard strains. Specifically, Minimum Inhibitory Concentration was obtained by diluting the test compounds according to a double dilution method, dispersing them in Mueller-Hinton agar medium, inoculating each of the test strains having 10⁷ cfu (colony forming unit) per ml in an amount of 2 μl to the medium and then incubating them at 37° C. for 20 hours. The results are shown in the following Table 1. From the results of Minimum Inhibitory Concentration test, it can be seen that the compound according to the present invention has a good activity against major pathogenic microorganisms, which cause hospital infection, including MRSA strains.

TABLE 1
Sensitivity test results using standard strains (μg/ml)

| | | | | S. | E. |
|---|---|---|---|---|---|
| | S. aureus | S. aureus | S. aureus | epidermidis | faecalis |
| | giorgio | 77 | K311 | Q033 | 2168 |
| I-1 | 0.13 | 0.25 | 0.5 | 0.063 | 0.5 |
| I-2 | 0.13 | 1 | 1 | 0.063 | 1 |
| I-3 | 0.13 | 0.5 | 0.5 | 0.063 | 0.5 |
| I-4 | 0.25 | 1 | 2 | 0.13 | 1 |
| I-5 | 0.25 | 1 | 2 | 0.13 | 0.25 |
| I-6 | 0.13 | 0.5 | 0.5 | 0.031 | 0.5 |
| I-10 | 0.5 | 2 | 2 | 0.063 | 4 |
| Vancomycin | 1 | 1 | 2 | 1 | >64 |

1. A compound represented by the following formula (1): 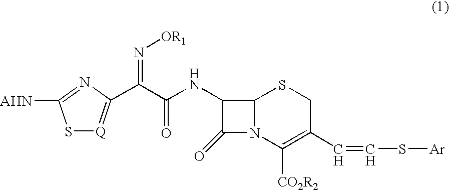
or pharmaceutically acceptable non-toxic salt, physiologically hydrolysable ester, hydrate, solvate or isomer thereof, in which
A represents hydrogen or amino-protecting group,
R₁ represents hydrogen, or represents C₁₋₆ alkyl, C₃₋₄ alkynyl, C₃₋₆ cycloalkyl or C₃₋₆ cycloalkyl-methyl, each of which may comprise one to three atoms selected from the group consisting of oxygen and halogen,
R₂ represents hydrogen or carboxyl-protecting group,
Ar represents 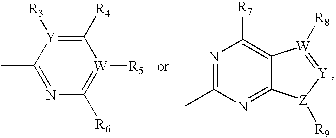
wherein
R₃, R₄, R₅ and R₇ independently of one another represent hydrogen; hydroxyl; C₁₋₆ alkyl; amino which is unsubstituted or substituted by C₁₋₆ alkyl; C₁₋₆ hydroxyalkyl; or C₁₋₆ alkylthio,
R₆ represents hydrogen; hydroxyl; amino which is unsubstituted or substituted by C₁₋₆ alkyl; C₁₋₆ alkyl; or 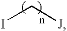
wherein
I represents S, NH, CH₂, or O,
n represents 0, 1, 2, 3, or 4,
J represents amino which is unsubstituted or substituted by C₁₋₆ alkyl; hydroxy; or C₁₋₆ alkoxy,
R₈ and R₉ independently of one another represent hydrogen; C₁₋₆ alkyl; C₁₋₆ alkylamino; hydroxy; or C₁₋₆ alkoxy,
W and Y independently of one another represent N or C, provided that R₃, R₅, R₈ do not exist when W or Y represents N,
Z represents CH or N,
Q represents CH, C—G, or N, wherein G represents halogen, and
the ethenyl group at C-3 position, to which heteroarylthio group is attached, may be present in the configuration of cis or trans.

2. The compound of claim 1, wherein G represents halogen selected from a group consisting of Cl and F; R₁ represents hydrogen, methyl, or cyclopentyl; and R₃, R₄, R₅ and R₇ independently of one another represent hydrogen, hydroxyl, or amino.

3. The compound of claim 1, wherein the compound is selected from a group consisting of the following:
I-1: (6R,7R)-7-{[2-(2-amino-5-chloro-1,3-thiazol-4-yl)-2-(hydroxyimino)acetyl]amino}-3-{(E)-2-[(6-amino-2-hydroxy-4-pyrimidinyl)sulfanyl]ethenyl}-8-oxo-5-thia-1-azabicyclo[4.2.0]oct-2-ene-2-carboxylic acid,
I-2: (6R,7R)-7-{[2-(2-amino-5-chloro-1,3-thiazol-4-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-3: (6R,7R)-7-{[2-(2-amino-5-chloro-1,3-thiazol-4-yl)-2-(hydroxyimino)acetyl]amino}-3-{(E)-2-[(2-amino-6-hydroxy-4-pyrimidinyl)sulfanyl]ethenyl}-8-oxo-5-thia-1-azabicyclo[4.2.0]oct-2-ene-2-carboxylic acid,
I-4: (6R,7R)-7-{[2-(2-amino-5-chloro-1,3-thiazol-4-yl)-2-(hydroxyimino)acetyl]amino}-3-{(E)-2-({2-[(2-aminoethyl)sulfanyl]-4-pyrimidinyl}sulfanyl)ethenyl}-8-oxo-5-thia-1-azabicyclo[4.2.0]oct-2-ene-2-carboxylic acid,
I-5: (6R,7R)-7-{[2-(2-amino-5-chloro-1,3-thiazol-4-yl)-2-(hydroxyimino)acetyl]amino}-(E)-2-({2-[(2-aminoethyl)sulfanyl]-6-methyl-4-pyrimidinyl}sulfanyl)ethenyl)-8-oxo-5-thia-1-azabicyclo[4.2.0]oct-2-ene-2-carboxylic acid,
I-6: (6R,7R)-7-{[2-(2-amino-5-chloro-1,3-thiazol-4-yl)-2-(hydroxyimino)acetyl]amino}-3-{(E)-2-[(4-amino-1H-pyrazolo[3,4-d]pyrimidin-6-yl)sulfanyl]ethenyl}-8-oxo-5-thia-1-azabicyclo[4.2.0]oct-2-ene-2-carboxylic acid,
I-7: (6R,7R)-3-[(E)-2-({6-amino-2-[(2-aminoethyl)sulfanyl]-4-pyrimidinyl}sulfanyl) ethenyl]-7-{[2-(2-amino-5-chloro-1,3-thiazol-4-yl)-2-(hydroxyimino)acetyl]amino}-8-oxo-5-thia-1-azabicyclo[4.2.0]oct-2-ene-2-carboxylic acid,
I-8: (6R,7R)-7-{[2-(2-amino-1,3-thiazol-4-yl)-2-(methoxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-9: (6R,7R)-7-{[2-(2-amino-1,3-thiazol-4-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-10: (6R,7R)-7-({2-(2-amino-5-chloro-1,3-thiazol-4-yl)-2-[(cyclopentyloxy)imino] acetyl}amino)-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-11: (6R,7R)-3-{(E)-2-[(2-amino-6-hydroxy-4-pyrimidinyl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-12: (6R,7R)-3-{(E)-2-[(6-amino-2-hydroxy-4-pyrimidinyl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-13: (6R,7R)-7-{[2-(S-amino-1,2,4-thiadiazol-3-yl)-2-(hydroxyimino)acetyl]amino}-3-{(E)-2-[(2,6-diamino-4-pyrimidinyl)sulfanyl]ethenyl}-8-oxo-5-thia-1-azabicyclo[4.2.0]oct-2-ene-2-carboxylic acid,
I-14: (6R,7R)-3-[(E)-2-({6-amino-2-[(2-aminoethyl)sulfanyl]-4-pyrimidinyl}sulfanyl) ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid,
I-15: (6R,7R)-3-[(E)-2-({2-[(2-aminoethyl)sulfanyl]-6-methyl-4-pyrimidinyl}sulfanyl) ethenyl]-7-{[2-(5-amino-1,2,4-thiadiazol-3-yl)-2-(hydroxyiminoacetyl)amino]-8-oxo-5-thia-1-azabicyclo[4.2.0]oct-2-ene-2-carboxylic acid,
I-16: (6R,7R)-3-[(E)-2-({2-[(2-aminoethyl)sulfanyl]-4-pyrimidinyl}sulfanyl)ethenyl]-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid, and
I-17: (6R,7R)-3-{(E)-2-[(4-amino-1H-pyrazolo[3,4-d]pyrimidin-6-yl)sulfanyl]ethenyl}-7-{[2-(5-amino-1,2,4-thiadiazol-3-yl)-2-(hydroxyimino)acetyl]amino}-8-oxo-5-thia-1-azabicyclo[4.2.0]oct-2-ene-2-carboxylic acid.

4. A process for preparing the compound of formula (1) according to claim 1, which comprises reacting a compound of the following formula (4): 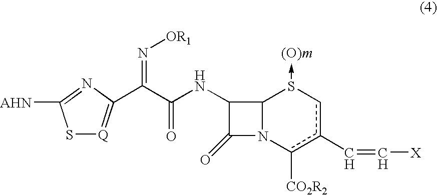
wherein A, R₁, R₂ and Q are as defined in claim 1, X represents halogen or sulfonyloxy, m represents 0 or 1, and the double bond at C-3 position may be present in the configuration of cis or trans, with a compound of the following formula (5):
HS—Ar (5)
wherein Ar is as defined in claim 1, in a solvent to produce a compound of the following formula (1a): 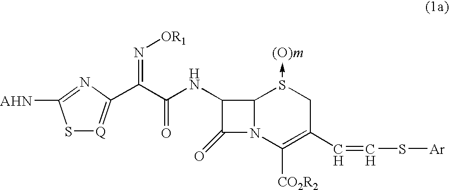
wherein A, R₁, R₂, Ar, Q, and m are as defined above, and the double bond at C-3 position may be present in the configuration of cis or trans, or reducing S→ oxide of the compound of formula (1a) wherein m is 1.

5. A process for preparing the compound of formula (1) according to claim 1, which comprises subjecting a compound of the following formula (13): 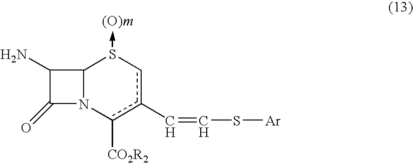
wherein Ar and R₂ are as defined in claim 1, and m represents 0 or 1, and the double bond at C-3 position may be present in the configuration of cis or trans, to an amide-bond forming reaction with a compound of the following formula (7): 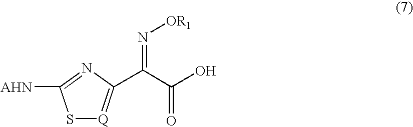
wherein A, R₁ and Q are as defined in claim 1, or a derivative thereof activated at its carboxyl group in a solvent to produce a compound of the following formula (1a): 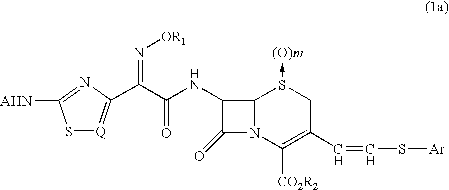
wherein A, R₁, R₂, Ar, Q, and m are as defined above, and the double bond at C-3 position may be present in the configuration of cis or trans, or reducing S→ oxide of the compound of formula (1a) wherein m is 1.

6. An antibacterial composition comprising the compound of formula (1) according to claim 1, as an active ingredient, together with a pharmaceutically acceptable carrier.

7. The antibacterial composition of claim 6 formulated into an oral preparation comprising the compound of formula (1) in an amount of 50 to 1,500 mg.
